(19) United States
(12) Patent Application Publication
(10) Pub. No.: US 2024/0212516 A1
(43) Pub. Date: Jun. 27, 2024

(54) DIRECT LARYNGOSCOPE TRAINING MODE FOR VIDEO LARYNGOSCOPE

(71) Applicant: Covidien LP, Mansfield, MA (US)

(72) Inventors: Bo Chen, Louisville, CO (US); Derek S. Tata, Boulder, CO (US); Peter Douglas Colin Inglis, Boulder, CO (US); Patrick J. Nellis, Georgetown (CA)

(73) Assignee: Covidien LP, Mansfield, MA (US)

(21) Appl. No.: 18/541,299

(22) Filed: Dec. 15, 2023

Related U.S. Application Data
(60) Provisional application No. 63/435,116, filed on Dec. 23, 2022.

(51) Int. Cl. G09B 5/02; A61B 1/00; A61B 1/05; A61B 1/06; A61B 1/267

(52) U.S. Cl. CPC G09B 5/02; A61B 1/00052; A61B 1/05; A61B 1/0676; A61B 1/267

(57) ABSTRACT

A training mode for a video laryngoscope is disclosed. The training mode may allow an operator to practice and maintain DL skills with a video laryngoscope. In the training mode, a video image typically provided on an integrated display of the video laryngoscope may be temporarily obscured, filtered, or turned off. Additionally, the unfiltered video image acquired by the video laryngoscope may be displayed on an external display, separate from the video laryngoscope. The external display may be viewable by a trainer (e.g., an attending) for teaching purposes. The training mode may also include an adjustment of a brightness and/or intensity of a light source on the video laryngoscope (e.g., a light source may be brighter/more intense during training mode than during normal video laryngoscopy). In an instance where the video is recorded, at least a portion of the recording that is associated with the training mode may be labeled.

### CROSS-REFERENCE TO RELATED APPLICATIONS

This application claims the benefit of U.S. Provisional Application No. 63/435,116 filed Dec. 23, 2022, entitled “Direct Laryngoscope Training Mode for Video Laryngoscope,” which is incorporated herein by reference in its entirety.

### INTRODUCTION

Laryngoscopes are commonly used during intubation of a patient (e.g., an insertion of an endotracheal tube into a trachea of the patient). Intubation via a laryngoscope may be performed under direct visualization of the larynx (e.g., direct laryngoscopy (DL)) or with indirect visualization (e.g., indirect laryngoscopy (IL)). One form of indirect laryngoscopy is video laryngoscopy (VL). In video laryngoscopy a medical professional (e.g., a doctor, therapist, nurse, clinician, or other practitioner) views a video image of the patient's larynx on a display screen. A video laryngoscope used to perform VL may include an integral display that is in the line-of-sight of the laryngoscope operator so that the patient airway and larynx is viewable on the display screen in real time to facilitate navigation and insertion of tracheal tubes within the airway.

It is with respect to this general technical environment that aspects of the present technology disclosed herein have been contemplated. Furthermore, although a general environment is discussed, it should be understood that the examples described herein should not be limited to the general environment identified herein.

### SUMMARY

Among other things, aspects of the present disclosure include systems and methods for a direct laryngoscope mode (e.g., training mode) for a video laryngoscope. In an aspect, the technology relates to a method for operating a video laryngoscope in a direct laryngoscopy training mode. The method includes acquiring images using a camera of the video laryngoscope; receiving an indication to operate the video laryngoscope in a direct laryngoscopy mode; in response to receiving the indication to operate the video laryngoscope in the direct laryngoscopy training mode, applying displaying obscured acquired images on an integrated display of the video laryngoscope; receiving an indication to operate the video laryngoscope in a video laryngoscopy mode; and in response to receiving the indication to operate the video laryngoscope in the video laryngoscopy mode, displaying, on the integrated display of the video laryngoscope, the acquired images.

In an example, the obscured acquired images include the acquired images and one or more of the following modifications: a blur; a watermark; or overlaid text. In another example, the method further includes, in response to receiving the indication to operate the video laryngoscope in the video laryngoscopy mode, reducing the brightness of the light source. In yet another example, the indication to operate the video laryngoscope in the video laryngoscopy mode is any input at the integrated display of the video laryngoscope. In still another example, the method further includes recording the acquired images in a recording; and labelling at least a portion of the recording as a training session. In still yet another example, the method further includes displaying, at the integrated display of the video laryngoscope, a torque indicator associated with an amount of torque detected by the video laryngoscope.

In another aspect, the technology relates to a video laryngoscope training mode system. The system includes an external display; and a video laryngoscope that includes an integrated display; a camera that acquires images while the video laryngoscope is powered on; a processor that operates to: receive an indication to operate the video laryngoscope in a direct laryngoscopy training mode; in response to receiving the indication to operate the video laryngoscope in the direct laryngoscopy training mode, display obscured acquired images on an integrated display of the video laryngoscope; and transmit the acquired images to the external display for unobscured display of the acquired images, concurrently with displaying the filtered images at the integrated display.

In an example, the video laryngoscope further comprising a light source with an adjustable brightness. In a further example, the processor further operates to increase the brightness of the light source in response to receiving the indication to operate the video laryngoscope in the direct laryngoscopy training mode. In another example, the video laryngoscope further includes a torque sensor; and the processor further operates to: acquire torque data from the torque sensor; and transmit the torque data to the external display for display. In yet another example, the acquired images are stored in memory of the video laryngoscope, and wherein at least a portion of the acquired images are labeled a training session.

In another aspect, the technology relates to a video laryngoscope that includes a power switch that powers the video laryngoscope on and off in response to user input; a handle portion; a display screen coupled to the handle portion; a blade portion, coupled to the handle portion, configured to be inserted into a mouth of a patient; a camera, positioned at a distal end of the blade portion, that acquires images while the video laryngoscope is powered on; a light source positioned at the distal end of the blade portion; a processor that operates to: activate a direct laryngoscopy training mode; receive acquired images from the camera; in response to activating the direct laryngoscopy training mode: display obscured acquired images on the display screen; and increase a brightness or intensity of the light source.

In an example, the processor operates to: transmit the acquired images to an external display for unobscured display of the acquired images. In another example, activating the direct laryngoscopy training mode is based on receiving a long press of the power button. In yet another example, while in the direct laryngoscopy training mode, the processor operates to display, on the display screen, unobscured device information.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1A shows a schematic of a patient environment including a video laryngoscope in a video laryngoscopy (VL) mode.

FIG. 1B shows a schematic of a patient environment including the video laryngoscope of FIG. 1A in a direct laryngoscopy (DL) training mode and an external display screen.

FIG. 2 shows a perspective view of a video laryngoscope.

FIG. 3 shows a block diagram of components of a video laryngoscope.

FIGS. 4A-4D show example display screens of a video laryngoscope in a training mode.

FIG. 5 shows a flow diagram of example user interfaces on display screens on a display of a video laryngoscope operating in a training mode.

FIG. 6 shows an example method for a training mode of a video laryngoscope.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, laryngoscopes are commonly used during intubation of a patient (e.g., an insertion of an endotracheal tube into a trachea of the patient). During intubation, the patient's airway and larynx may be visualized via direct laryngoscopy (DL) or indirect laryngoscopy (IL). One form of indirect laryngoscopy is video laryngoscopy (VL), which includes displaying a video image of the patient's larynx on a display screen. The video image displayed during VL during an intubation procedure may assist a medical professional to visualize the patient's airway and facilitate manipulation and insertion of the tracheal tube with the video laryngoscope. The display screen may be integrated into a video laryngoscope in a direct line-of-sight of the medical professional operating the video laryngoscope. Additionally or alternatively, a display screen may be a larger, external display separate from the housing of the video laryngoscope. An external display may allow other medical professionals participating in the medical procedure to track the progress of the intubation or other airway procedure in real time.

Medical professionals may use direct laryngoscopy, video laryngoscopy, or both, during an intubation procedure. When using a video laryngoscope, some operators primarily use direct visualization, some primarily use the video image, and other shift between both. There is not a standardized clinical practice or guidelines for visualization during intubation. Instead, each operator may use different visualization patterns (e.g., when and how to visualize the patient's airway/larynx and video image). Although most medical professionals have access to a video laryngoscope to perform VL, most medical professionals agree that maintaining DL skills is important. Some medical professionals believe that direct laryngoscopy may result in better patient outcomes. Additionally, a video laryngoscope may not always be available or accessible. Even when a video laryngoscope is available, DL is the fallback in an event of equipment failure, excess bodily fluid, or other event that results in interference with obtaining or displaying a video image. DL may be performed using a video laryngoscope simply by having an operator not view the display screen. This may be challenging, however, when the display is integrated into the video laryngoscope and in direct line-of-site of the operator. Other challenges of using a video laryngoscope during DL may include poor lighting and excess blade curvature interfering with direct visualization of the larynx.

Provided herein is a training mode for a video laryngoscope to allow an operator to practice and maintain DL skills with a video laryngoscope. In a direct laryngoscopy mode (otherwise referred to herein as a training mode, DL mode, direct laryngoscopy training mode, or DL training mode), a video image typically provided on an integrated display of the video laryngoscope may be temporarily obscured, filtered, masked, or turned off. Additionally, the video image acquired by the video laryngoscope may be displayed on an external display, separate from the video laryngoscope. The training mode may also include adjustment of a brightness and/or intensity of a light source. For example, during a training mode, brightness and/or intensity may be increased relative to a typical brightness and intensity of video laryngoscopy (e.g., to reduce glare and/or internal reflection for the camera). In an example where video is obscured at integrated display of the video laryngoscope while displayed (e.g., unmasked and unobscured) at an external display, the external display may be viewable by a trainer (e.g., an attending) for teaching purposes. In an instance where the video is recorded, at least a portion of the recording that is associated with the training mode may be tagged or labeled.

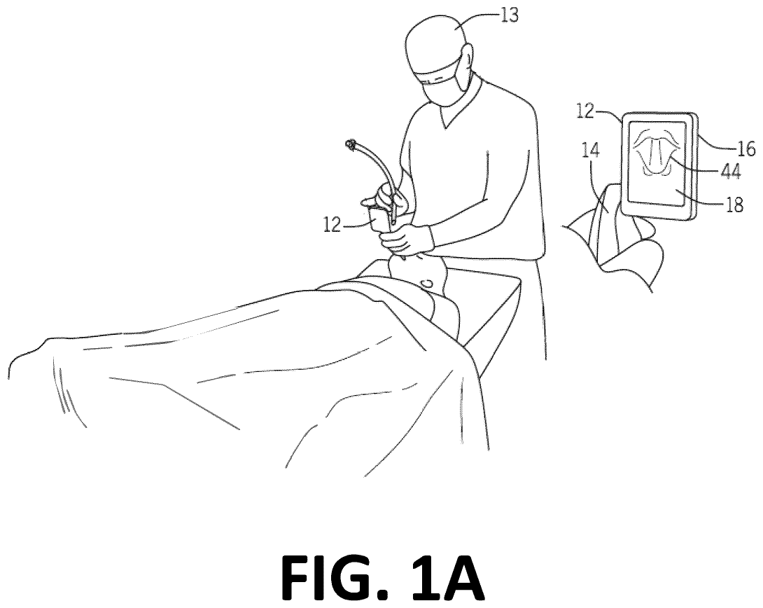

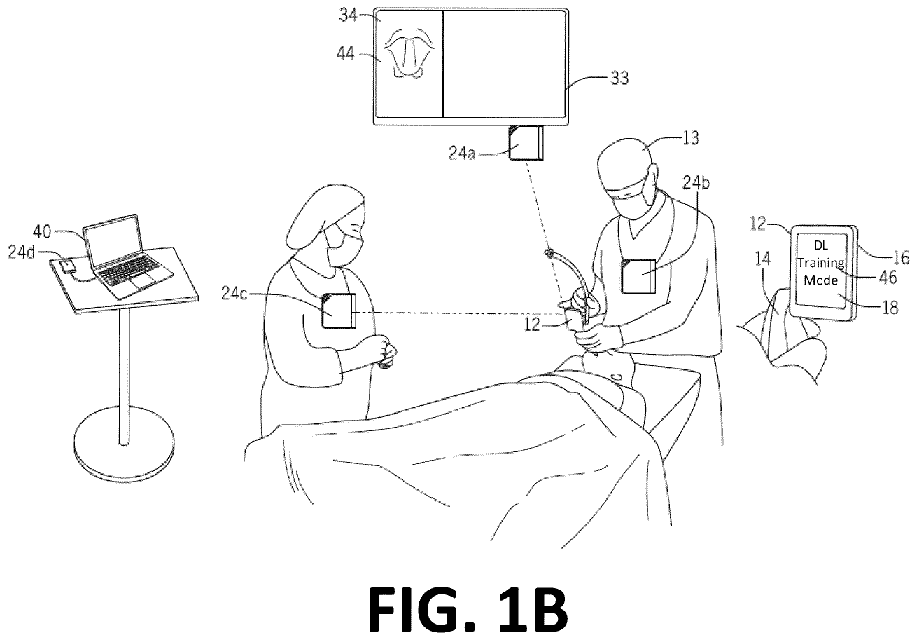

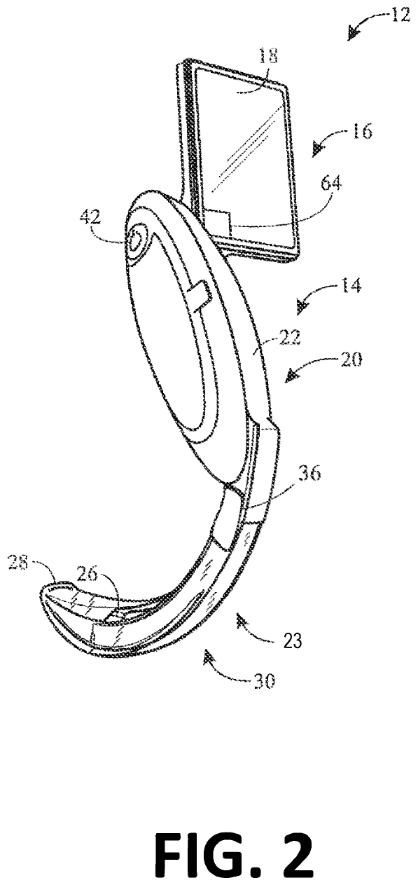

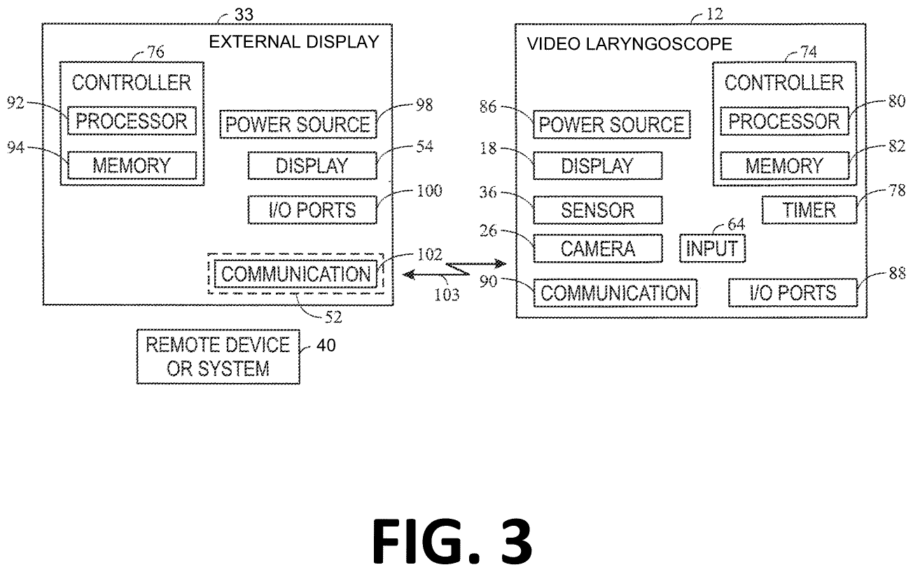

FIGS. 1A-1B show schematics of patient environments for a video laryngoscope. Specifically, FIG. 1A shows a schematic of a patient environment including a video laryngoscope 12 in a video laryngoscopy (VL) mode. FIG. 1B shows a schematic of a patient environment including the video laryngoscope 12 of FIG. 1A in a direct laryngoscopy (DL) training mode and an external display screen 33. The patient environment may be any room where an intubation is being performed, such as a medical suite in a hospital or other care setting, an operating or other procedure room, patient recovery room, an emergency intubation setting, or other environments. As described herein, the video laryngoscope 12 may be used for airway visualization of a patient in a VL mode or DL training mode. Aspects of the video laryngoscope are further shown in FIGS. 2-3.

As shown in FIGS. 1A-1B, a laryngoscope operator 13 (otherwise referred to herein as a trainee 13 or medical professional 13) holds a handle portion of the body 14 of the video laryngoscope 12 coupled to a display portion 16 having a display screen 18. The video laryngoscope 12 may be used as part of an intubation procedure to advance a tracheal tube into a patient airway to secure the airway for mechanical ventilation. Accordingly, the operator 13 of the video laryngoscope 12 performs the intubation and directly manipulates the endotracheal tube within the patient's airway. Other clinicians in the patient environment may assist the laryngoscope operator 13, monitor a condition of the patient, prepare or adjust medical equipment in the patient environment, and/or wait until the airway is secured to perform other procedures or interventions. Additionally or alternatively, a trainer (e.g., an attending or experienced medical professional) may be present to instruct, teach, oversee, review, critique or otherwise facilitate operation of the video laryngoscope 12 by the operator 13 (e.g., trainee 13).

Video images 44 may be acquired by the video laryngoscope 12 during operation. Depending on the mode of operation of the video laryngoscope 12, the acquired images 44 are displayed on one or more display screens. For example, if the video laryngoscope 12 is operating in a VL mode (e.g., as shown in FIG. 1A), the acquired images 44 may be displayed at an integrated display 18 (e.g., laryngoscope display 18) of the video laryngoscope 12 in the line of sight of the operator 13.

Alternatively, if the video laryngoscope 12 is operating in a DL training mode (e.g., an example of which is shown in FIG. 1B), the acquired images 44 may be streamed to and displayed at an external display 33 in substantially real time (shown in FIG. 1B) while modified or obscured images 46 may be displayed at the integrated display 18 in substantially real time. The modified images 46 may be a filtered, obscured, hidden, masked, or otherwise modified version of the acquired images 44. Alternatively, in the DL training mode, the acquired images 44 may not be displayed in any manner on the laryngoscope display 18.

As described above, the acquired images 44 may be displayed at an external display 33. Thus, the laryngoscope 12 may be configured to communicate with an external display 33 and/or other remote devices 40 or systems via any of a variety of techniques. When the video laryngoscope 12 and the external display 33 are communicatively coupled to one another, an indication may be provided on the laryngoscope display 18. Communication between the video laryngoscope 12 and the external display 33 may be facilitated by one or more communication devices (e.g., wireless transceivers or hubs, which may be a wireless adapter, dongle, bridge device, etc.) that are configured to establish wireless communication with one another using any suitable protocol. In an example, an adapter may be coupled to the external display 33 (e.g., by plugging the wireless adapter into a Universal Serial Bus (USB) port of the external display 33) to relay information or commands between the video laryngoscope 12 and the external display 33. The wireless adapter may be movable between multiple external displays.

In another example, as shown in FIG. 1B, display of the acquired images 44 at an external display 33 may use wireless hubs 24a, 24b, 24c to stream the acquired images 44 from the video laryngoscope 12 to a first wireless hub 24a, to a second wireless hub 24b, 24c, and provided to the external display 33 for display on all or a portion of an external display screen 34. Examples of using wireless hubs to stream or display acquired images 44 at devices other than the video laryngoscope 12 are described in at least U.S. Provisional Patent Application 63/257,895, titled “Video Laryngoscope Image File Management Systems and Methods,” filed on Oct. 20, 2021, the entirety of which is hereby incorporated by reference. Although wireless hubs 24 are shown in FIG. 1B, any other form of communication between the video laryngoscope 12 and an external display 33 capable of substantially real time streaming is contemplated, such as wired or wireless transceivers. The video laryngoscope 12 and/or the external display 33 may include ports (e.g., USB ports, Ethernet ports, high-definition multimedia interface (HDMI) ports, optical ports, infrared ports, near field ports, or the like) that enable a wired connection.

The acquired images 44 may be stored in a memory 82 of the video laryngoscope 12. Stored acquired images may be transferred (e.g., via wireless hubs 24a, 24c) to a remote device 40 (e.g., a computer). The stored images may be reviewed, edited, or otherwise interacted with at the remote device 40. In some examples, the stored images may also be reviewed or replayed on the display screen of the video laryngoscope 12 itself. For instance, to help enhance training, stored images such as prior video files, may be displayed on the video laryngoscope 12.

The stored images may be associated with a tag, label, or other metadata depending on the mode of operation of the video laryngoscope 12. For example, a recording (e.g., a sequence of stored, acquired images) may be tagged or labeled with a training label if the video laryngoscope was operating in a DL training mode at the time some or all of the acquired images were captured. The tag or label may be associated with all or a portion of the recording, may be per frame or per image, or may indicate a timestamp or timestamp ranges at which the acquired images were captured while the video laryngoscope was operating in DL training mode. The tag or label may also include a watermark on the training images to indicate which images were acquired during DL mode. Tagged or labeled images may be saved in a different folder associated with training images. Additionally or alternatively, tagged or labeled images may include a different naming convention for the training images. As an example, the file naming convention may be TRAIN_{FILE NAME}.mp4. A training-session label or tag may also or alternatively be provided in the metadata of the video file.

FIG. 2 shows a perspective view of a video laryngoscope 12. As shown, the video laryngoscope 12 has a body 14 (e.g., reusable body). The body 14 includes a display portion 16 having a display screen 18 that is configured to display images and/or other data, a handle portion 20 having a handle 22 that is configured to be gripped by the medical professional during the laryngoscopy procedure, and an elongate portion or arm 23 that supports a camera 26 and light source (e.g., light-emitting diodes (LEDs)) that is configured to obtain images (e.g., acquired images 44, which may be still images and/or moving images, such as videos). The camera 26 and light source may be incorporated on the distal end of the arm 23. The light source may be provided as part of the camera 26 or separate from the camera 26 on the blade 28 or arm 23.

The light source may have an adjustable brightness and/or intensity. In an example, DL may have an industry standard for brightness and/or intensity of light emitted by a laryngoscope during a DL procedure. The industry standard for brightness and/or intensity may be different from a desired brightness and/or intensity for use with a camera during VL. For instance, brightness and/or intensity during VL may be less bright and/or less intense than during DL to reduce glare and/or reflection issues with the camera of the video laryngoscope. A brightness and/or intensity of lighting may be the same between different modes of operation of a video laryngoscope (e.g., DL mode and VL mode) that meets both the industry standard for DL without undue interference with the camera acquiring images during VL.

In examples, the display portion 16 and the handle portion 20 may not be distinct portions, such that the display screen 18 is integrated into the handle portion 20. In the illustrated embodiment, an activating cover, such as a removable laryngoscope blade 28 (e.g., activating blade; disposable cover, sleeve, or blade), is positioned about the arm 23 of the body 14 of the laryngoscope 12. Together, the arm 23 of the body 14 and the blade 28 form an insertable assembly 30 that is configured to be inserted into the patient's oral cavity. It should be appreciated that the display portion 16, the handle portion 20, and/or the arm 23 that form the body 14 of the laryngoscope 12 may be fixed to one another or integrally formed with one another (e.g., not intended to be separated by the medical professional during routine use) or may be removably coupled to one another (e.g., intended to be separated by the medical professional during routine use) to facilitate storage, use, inspection, maintenance, repair, cleaning, replacement, or interchangeable parts (e.g., use of different arms or extensions with one handle portion 20), for example.

The handle 22 and/or arm 23 may include one or more sensors 36 capable of monitoring functions (e.g., different, additional, and/or advanced monitoring functions). The sensors 36 may include a torque sensor, force sensor, strain gauge, accelerometer, gyroscope, magnet, magnetometer, proximity sensor, reed switch, Hall effect sensor, etc. disposed within or coupled to any suitable location of the body 14. The sensors 36 may detect interaction of the video laryngoscope 12 with other objects, such as a blade 28 or physiological structures of the patient (e.g., teeth, tissue, muscle, etc.).

Information received at the sensor(s) may be displayed at the integrated laryngoscope display 18 and/or at an external display 33. In an example, one or more sensors of the video laryngoscope 12 may measure force or pressure being asserted on a patient by a portion of the video laryngoscope 12. For instance, a strain gauge on a hinge, a gyroscope, and/or an accelerometer may be used to determine a load exerted (e.g., load on teeth of the patient) and/or a torque exerted (e.g., torque at the video laryngoscope 12 from pressure applied at patient's neck or throat). Sensor measurements may be used to enhance patient safety and otherwise avoid or reduce trauma to the patient by displaying sensor information and/or alerts or warnings when sensor readings are undesirable (e.g., if a load or torque is too high). For instance, a torque indicator and/or load indicator may be displayed at an integrated display of the video laryngoscope 12 and/or an external display associated with an amount of torque and/or load detected. Additionally or alternatively, examples of sensors with monitoring functions based on a magnet and/or magnetometer are described in at least U.S. Pat. No. 11,033,180, titled “Video Laryngoscope Systems and Methods,” filed on Oct. 29, 2018, the entirety of which is hereby incorporated by reference.

The laryngoscope 12 may also include a power button 42 that enables a medical professional to power the laryngoscope 12 off and on. The power button 42 may also be used as an input device to access settings of the video laryngoscope 12, including a mode of operation (e.g., DL training mode or VL mode). Additionally, the video laryngoscope 12 may include an input button, such as a touch or proximity sensor 64 (e.g., capacitive sensor, proximity sensor, or the like) that is configured to detect a touch or object (e.g., a finger or stylus). The touch sensor 64 may enable the medical professional operating the video laryngoscope 12 to efficiently provide inputs or commands, such as inputs to select a mode of operation (e.g., VL mode or DL mode), inputs that cause the camera 26 to obtain or store an image on a memory of the laryngoscope, and/or any other inputs relating to function of the video laryngoscope 12.

FIG. 3 shows a block diagram of components of the video laryngoscope 12 and external display 33. As shown, the video laryngoscope 12 and/or the external display 33 may include various components that enable the video laryngoscope 12 to carry out the techniques disclosed herein. For example, the video laryngoscope 12 may include the display screen 18, the camera 26, a light source (e.g., which may integrated into the camera or separate from the camera), sensor(s) 36, and input (e.g., touch sensor) 64, as well as a controller 74 (e.g., electronic controller), one or more processors 80, a hardware memory 82, a power source (e.g., battery) 86, input/output (I/O) ports 88, a communication device 90, and a timer 78. In some embodiments, the timer 78 may track relative time (e.g., a start time, an end time, a length of the monitoring session), which may be communicated to and/or used by the external display 33 (e.g., in combination with data from a real-time clock of the external display 33), such as to determine and/or associate a real time with the images obtained by the laryngoscope 12. In an example, the timer 78 may track timestamps and/or tags or labels at times in a monitoring session or recording at which a mode of the video laryngoscope changes (e.g., from DL training mode to VL mode or vice versa).

The external display 33 may include a display screen 54, a controller 76 (e.g., electronic controller), one or more processors 92, a hardware memory 94, a power source (e.g., battery or input from external power source) 98, I/O ports 100, and a communication device 102. The power sources 86, 98 may be rechargeable and/or replaceable batteries. The communication devices 90, 102 may be wireless transceivers that are configured to establish wireless communication 103 with one another. By way of example, the communication devices 90, 102 may be configured to communicate using the IEEE 802.15.4 standard, and may communicate, for example, using ZigBee, WirelessHART, or MiWi protocols. Additionally or alternatively, the communication devices 90, 102 may be configured to communicate using the Bluetooth standard or one or more of the IEEE 802.11 standards. As noted above, in some embodiments, the communication device 90 may be provided in the form of adapters or wireless hubs 24 that may be configured to couple to the video laryngoscope 12 and/or external display 33 to facilitate wireless communication 103 between the video laryngoscope 12 and the external display 33.

Additionally or alternatively, the video laryngoscope 12 may communicate with one or more remote devices 40, such as computing systems (e.g., handheld or portable computing systems operated by the medical professional, such as a tablet, smart phone, or the like; hospital computing systems; or the like), room display systems, and/or hospital data storage systems. The remote devices 40 may be configured to communicate (e.g., send and/or receive signals from) with the video laryngoscope 12 and/or the external display 33 via wireless or wired connections. For example, the video laryngoscope 12 and/or the external display 33 may relay acquired images 44 and/or modified images 46 to the remote device 40 for display, storage, and/or other interaction.

In some embodiments, the laryngoscope 12 and the external display 33 include electrical circuitry configured to process signals, such as signals generated by the camera 26 or light source, signals generated by the sensor(s) 36, and/or control signals provided via inputs, such as the inputs 58, 60 of the external display 33, or the input touch sensor 64 on the video laryngoscope 12. The processors 80, 92 may be used to execute software. For example, the processor 80 of the video laryngoscope 12 may be configured to receive signals from the camera 26 and light source and execute software to generate an image, adjust a brightness and/or intensity of the light source, and/or to carry out any of a variety of processes in accordance with the present disclosure (e.g., display acquired images 44, modify the acquired images, display modified images 46, store acquired images or modified images, transfer images, tag or label images, etc.).

The processors 80, 92 may include multiple microprocessors, one or more “general-purpose” microprocessors, one or more special-purpose microprocessors, and/or one or more application specific integrated circuits (ASICS), or some combination thereof. For example, the processors 80, 92 may include one or more reduced instruction set (RISC) processors. It should be appreciated that the various processing steps may be carried out by either processor 80, 92 or may be distributed between the processors 80, 92 in any suitable manner.

The hardware memory 82, 94 may include a volatile memory, such as random access memory (RAM), and/or a nonvolatile memory, such as read-only memory (ROM). It should be appreciated that the hardware memory 82, 94 may include flash memory, a hard drive, or any other suitable optical, magnetic, or solid-state storage medium, other hardware memory, or a combination thereof. The memory 82, 94 may store a variety of information and may be used for various purposes. For example, the memory 82, 94 may store processor-executable instructions (e.g., firmware or software) for the processors 80, 92 to execute, such as instructions for processing signals generated by the camera 26 to generate the image, provide the image on the display screen 18, and/or transmit the image to the monitor 50. The hardware memory 82, 94 may store data (e.g., acquired images, timestamps or other time data, tags or labels, mode data, etc.), instructions (e.g., software or firmware for generating the images, storing the images, transmitting the images, etc.), and any other suitable data.

The processor 80 of the video laryngoscope 12 may receive mode information about operation of the video laryngoscope 12, such as operation in DL mode or VL mode. The processor 80 of the video laryngoscope 12 may be configured to receive user inputs to modify display of images at the laryngoscope display 18, external display 33, or remote device 40. For example, the processor 80 may control display of acquired images 44 at the laryngoscope display 18 in a VL mode and control of display of modified images 46 at the laryngoscope display 18 in a DL training mode. The processor 80 of the video laryngoscope 12 may modify the acquired images 44 into modified images 46. Display of acquired images 44 and/or modified images 46 at an external display 33 or remote device 40 may also be controlled by the processor 80 of the video laryngoscope 12, based on the operation mode.

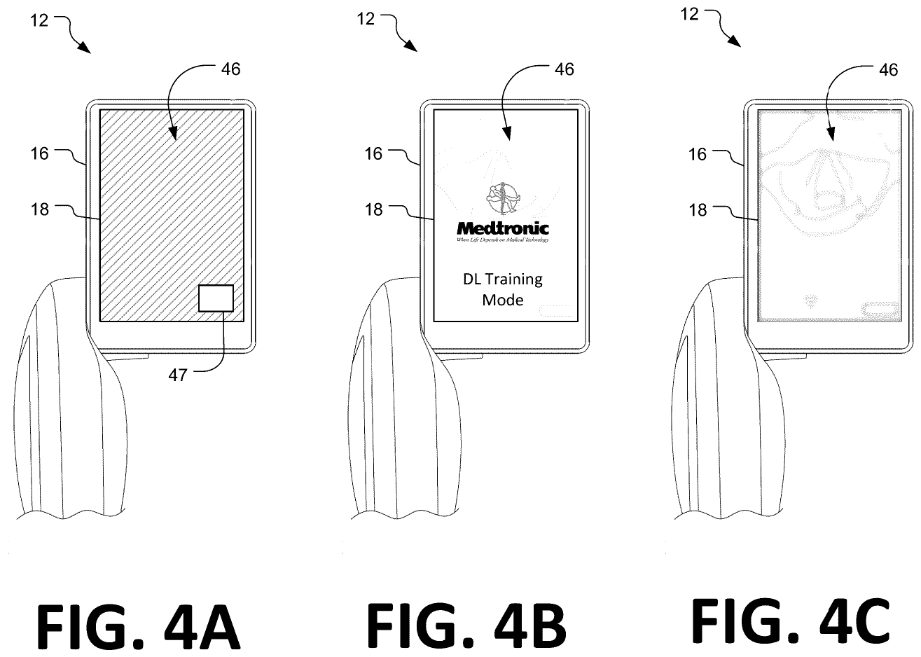

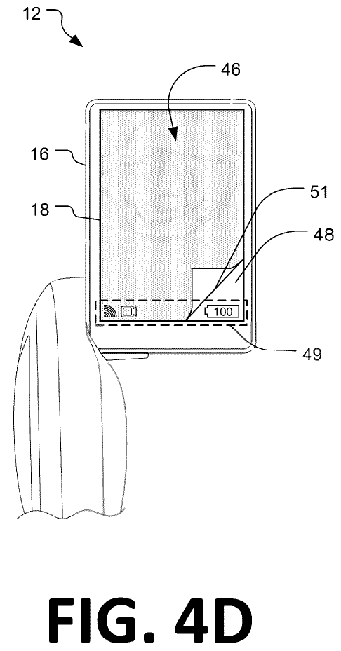

FIGS. 4A-4D show example display screens 18 of a video laryngoscope 12 in a DL training mode. When in the DL training mode, a display screen 18 of a display 16 of the video laryngoscope 12 may hide, filter, obscure, mask, or otherwise modify acquired images 44 in a way that a trainee 13 may no longer rely solely on the display screen 18 for intubation using the video laryngoscope 12. Different examples of modified images 46 are shown on the display screen 18 of FIGS. 4A-4D. For instance, FIG. 4A depicts hidden modified images 46 (e.g., a blank screen, solid screen, etc.). For instance, a blank or solid color layer may be overlaid on top of the acquired images 44 to form the modified images 46. In some examples, the unmodified acquired images 44 are still processed and effectively rendered behind the blank or solid layer (e.g., in a z-order direction), such that exiting the training mode almost immediately results in the unmodified acquired images 44 being displayed on the laryngoscope display 18.

FIG. 4B depicts text included in the modified images 46. Text may itself be the modified images 46 (e.g., text is the only image displayed) or text may be overlayed or watermarked. The text may indicate or state that the video laryngoscope 12 is operating in a DL training mode. FIG. 4C depicts masked acquired images for the modified images 46. Masking of acquired images may include changing or modifying characteristics of the acquired images, such as brightness, contrast, blur, compression, gamma, transparency, sharpness, noise, or other corrections. The masking may include providing or rendering a partially transparent layer in front of (e.g., front z-order) of the unmodified acquired images 44 to form the modified images 46.

FIG. 4D depicts modified images 46 that are, in part, masked and include at least a portion of the acquired images. For example, a mask may not be applied to an unmodified portion 48 of the acquired images. In an instance, the mask may appear as digitally peelable with a peeling indicator 51, with the unmodified portion 48 appearing as a portion of the acquired images that has been unobscured via a start of a digital peel-off of the mask. A touch or swipe input of the peeling indicator 51 inward from the edge may cause the unmodified acquired images 44 to be revealed and/or the DL training mode to be exited (e.g., change the video laryngoscope to a VL mode).

In each of the modified images 46 for the DL training mode, there may be some indication that the video laryngoscope 12 has an operable display screen 18. For example, if the modified images 46 hide the acquired images (e.g., the display screen 18 appears dark or appears to be powered off), there may be some indicator 47 on the display screen 18 that the video laryngoscope 12 is powered on. The indicator 47 may indicate that the video laryngoscope 12 is in a DL training mode and/or indicate that an input at video laryngoscope 12 may return the video laryngoscope to a VL mode. Additionally or alternatively, device information 49 may be displayed on the display screen 18 during operation in DL training mode. The device information 49 may be modified (e.g., behind the obfuscation mask in FIG. 4C) or unmodified (e.g., in front of the obfuscation mask in FIG. 4D) in the DL training mode. The device information 49 may include information about communication connections, recording options, storage space, battery life, sensor 36 readings, etc. For instance, the device information may include a power icon indicating remaining battery life and/or a connectivity icon indicating connection to an external device or display, among other icons. The indicator 47 and/or device information 49 may allow a user to easily determine that the video laryngoscope 12 is operable in VL mode, if desired.

Display of the modified images 46 may be removed or stopped to reveal the unobscured, unmasked acquired images based on an input at the video laryngoscope 12, such as a touch at the display screen 18 and/or a selection of the power button and/or any other user input. In an example, any and all touch inputs at the display screen 18 may cause the unobscured, unmasked acquired images to be displayed (e.g., exiting DL training mode and operating in VL mode). Alternatively, a touch input of a digital peel (e.g., swipe input) may result in display of the unobscured, unmasked acquired images.

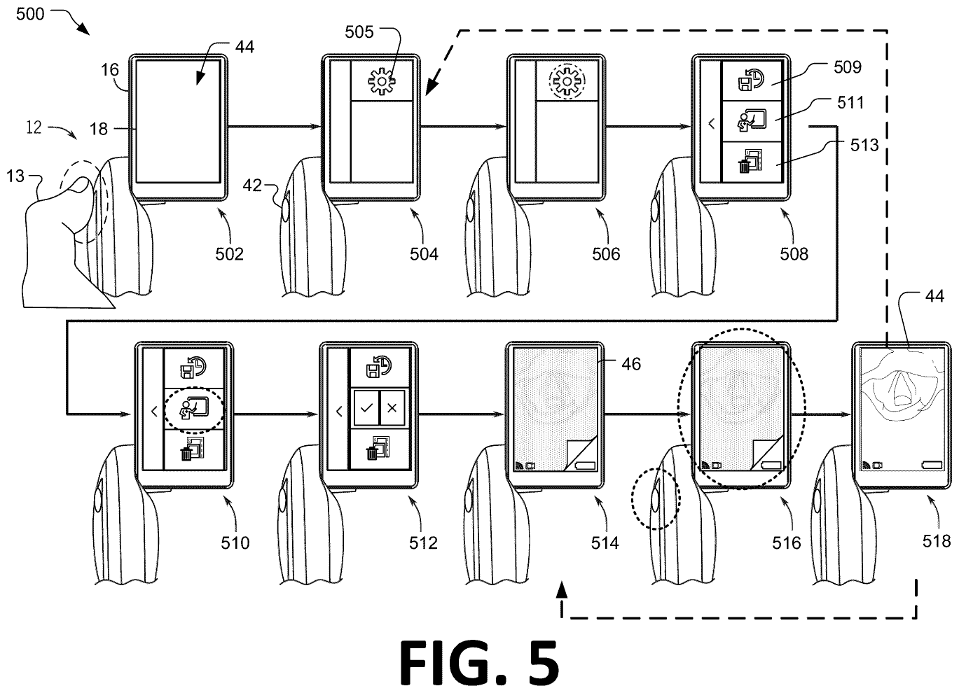

Reentering operation in DL training mode, after operating in VL mode, may involve a power off and power on of the video laryngoscope 12. This may prevent, or reduce the likelihood of, accidental re-entering into training mode in a situation where VL mode is being relied on or used for a medical procedure. Alternatively, the video laryngoscope 12 may return to operation in DL training mode (e.g., after exiting DL training mode into VL mode) in a similar manner to initial entry into DL training mode operation, such as that described below in FIG. 5. As another alternative, an operator 13 or trainee 13 may switch between VL mode and DL training mode with a predefined input (e.g., a specific touch input, such as a swipe up for VL and swipe down for DL training mode, a simple touch to switch, and/or a touch input and power button 42 input combination, etc.).

FIG. 5 shows a flow diagram 500 of example user interfaces 502-516 on a display 16 of a video laryngoscope 12 operating in a training mode. User interface 502 a blank screen or a screen that may be presented as the laryngoscope 12 powers on. The operator 13 may transition to one or more options screens, such as a settings screen 504. The information screen 504 may be accessed by a depression of a power button 42 for at least a threshold time (e.g., a long hold of the power button 42, such as for at least 5, 10, or more seconds). In an example, the information screen 504 may only be accessible during the duration of the long hold of the power button 42 (e.g., the information screen 504 may disappear to result in user interface 502 when the power button 42 is released). This may prevent an operator 13 from accidentally reaching or remaining on the information screen 504.

A settings option 505 may be selected at the information screen 504, such as the selection shown in user interface 506. Selection of the settings option may provide a settings menu 508. In the example shown in FIG. 5, the settings menu 508 includes three options. An upper icon 509, as shown, may be selectable to operate the video laryngoscope 12 in a recording mode. A middle icon 511, as shown, may be selectable to operate the video laryngoscope 12 in the training mode. A lower icon 513 may be selectable to delete retained recordings stored in memory of the video laryngoscope 12, such as bulk deletion or individual file selection.

A selection of the training mode icon 511 is shown in user interface 510. A confirmation screen 512 to confirm selection of the training mode may be provided. Selection and confirmation of the DL training mode in user interfaces 510-512 displays modified images 46 for the training mode, as otherwise described herein (and as depicted at least with respect to FIGS. 4A-4D). Selection of the DL training mode may cause automatic recording, streaming to an external device, and/or tagging or labeling of training images. For instance, when the video laryngoscope enters the DL training mode, recording of the video may automatically commence and/or streaming to the external device may automatically commence. In contrast, for non-training modes, such actions may not be automatically performed. Instead, in the non-training mode, the recording or streaming features may need to be selectively activated.

When a user input at the display screen 18 and/or power button 42 is received as shown in user interface 516, the video laryngoscope 12 exits the training mode (e.g., returns to the VL mode) and displays the unmodified acquired images 44, such as at user interface 518. A user input anywhere at or along the video laryngoscope (e.g., at the display screen 18, power button 42, or elsewhere) may terminate the DL training mode. In some examples, a maximum training time may be set, and when that maximum training time expires, the laryngoscope 12 exits the DL training mode.

As described above, exiting the DL training mode may or may not allow re-entry into the DL training mode until the laryngoscope 12 is powered off and powered on again. In an instance where re-entry into the DL training mode is allowable without a power cycle, user interface 518 may be returned to user interface 514 via an input (e.g., at user interface 516, such that the flow diagram may be bi-directional between user interfaces 514-518). As another alternative, after the selection at user interface 516, flow from user interface 516 to settings user interface 504 may be allowed.

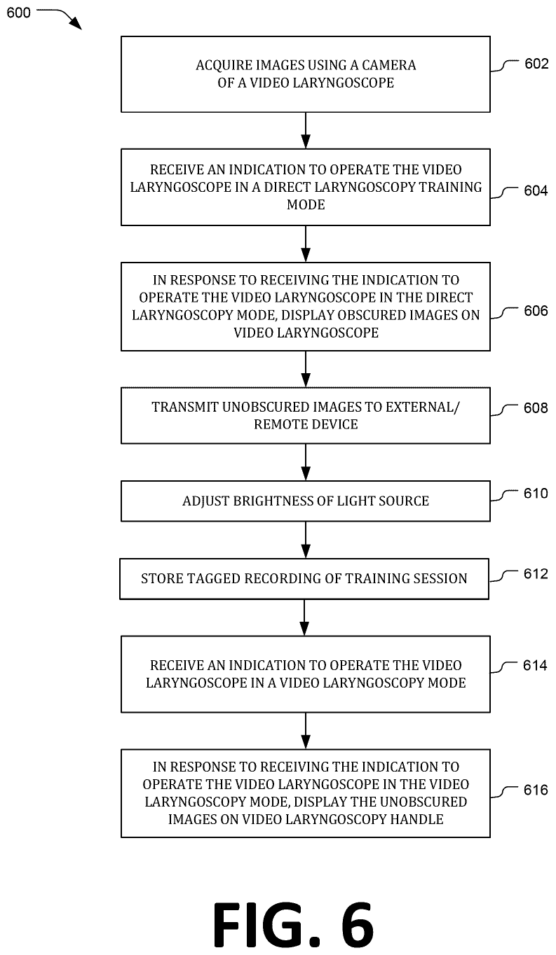

FIG. 6 shows an example method according to the disclosed technology. The example method includes operations that may be implemented or performed by the systems and devices disclosed herein. For example, the video laryngoscope 12, external display 33, and/or remote device 40 depicted in at least FIGS. 1A, 1B, 2, and 3 may perform the operations described in the methods. In addition, instructions for performing the operations of the methods disclosed herein may be stored in a memory of the video laryngoscope, external display, and/or remote device (e.g., system memories 82, 94 described in FIG. 3).

FIG. 6 shows an example method 600 for a training mode (e.g., direct laryngoscopy mode) of a video laryngoscope. At operation 602, images are acquired using a camera of a video laryngoscope. For example, when the video laryngoscope is powered on, images may automatically be captured by the video laryngoscope. A light source (e.g., in the camera or proximate the camera) may have an adjustable brightness and/or intensity. For example, a first brightness and/or intensity may be used to acquire images in a first mode of operation of the video laryngoscope and a second brightness and/or intensity may be used in a second mode of operation of the video laryngoscope. Acquired images may be recorded, stored, sent, streamed, modified (e.g., filtered, masked, obscured, hidden, or otherwise changed), etc.

At operation 604, an indication is received to operate the video laryngoscope in a direct laryngoscopy (DL) training mode. The indication may be a series of selections at the video laryngoscope, including selection at a power button and/or selection at a touch display integrated into the video laryngoscope. The indication may be similar to one or more selections described with respect to at least FIG. 5. In an example, the indication may include a long press of a power button of the video laryngoscope.

At operation 606, in response to receiving the indication to operate the laryngoscope in the DL training mode, modified or obscured acquired images are displayed on the display screen of video laryngoscope. Modification may include filtering, masking, obscuring, hiding, or otherwise changing one or more characteristics of the displayed images to prevent, or reduce the likelihood of, the images from being solely relied upon by an operator of the video laryngoscope during a medical procedure (e.g., intubation). In an example, all or a portion of the images may have a blur, transparency, varied contrast and/or brightness, text, overlaid text, etc. For example, an object or layer may be displayed in front of the acquired images to provide the modified (e.g., obscured) effect. The obscured images may be displayed at the integrated display of the video laryngoscope in substantially real time. For instance, an obscured video stream is displayed on the display of the video laryngoscope. Accordingly, the operator is still able to perceive that the video laryngoscope is functioning. The display screen may also include an indication of mode of operation (e.g., VL mode or DL mode) and/or device information (e.g., battery life, images captured, storage capacity, wireless connection, etc.), which may help remind an operator of the video laryngoscope that the display screen of the video laryngoscope is operational (e.g., not malfunctioning). At operation 608, the unmodified acquired images are transmitted to an external display and/or remote device for unmodified display of the acquired images. The unmodified acquired images may be displayed at the external display concurrently with display of the modified images at the integrated display. Display at the external device may be substantially in real time. For example, while the modified images are displayed on the laryngoscope, the unmodified acquired images and/or the modified images may be sent to an external display or external device for display separate from the video laryngoscope.

The display separate from the laryngoscope may be viewable by another medical professional during operation of the video laryngoscope by the operator (e.g., a trainer may view an external display showing the unmodified acquired images while, or concurrently with, a trainee operating the video laryngoscope in a DL training mode). The transmitted images may be tagged or labeled for display at the external display. For example, the acquired images displayed at an external device while the video laryngoscope is operating in a DL training mode may include an indication that an operator of the video laryngoscope is seeing obscured images at an integrated display of the video laryngoscope or any other indication that the video laryngoscope is operating in a DL training mode (e.g., a symbol, text, a watermark, side-by-side display of both the acquired images and the modified images, etc.).

Additional data may also be displayed locally on the video laryngoscope and/or transmitted to the external display. For instance, where additional sensors (e.g., strain gauges, accelerometers, etc.) are included in the laryngoscope, such sensor data may be displayed locally and/or at the external display. For example, a torque sensor may generate torque data, and torque indicator may be displayed. In an example, the additional sensor data is transmitted for display at the external display, but the additional sensor data is not displayed at the local display of the video laryngoscope. As such, the trainer or supervisor at the external display may be able to see additional information to help train or guide the operator of the laryngoscope.

At operation 610, a brightness of the light source may be adjusted based on entering the DL training mode. For example, in direct laryngoscopy, additional light at the distal end of the laryngoscope blade may be beneficial to illuminate the trachea, mouth, or other portions of the patient in which the laryngoscope is inserted to that the human eye can see such features. Further, because the camera is not being directly relied upon during direct laryngoscopy, glare issues resulting from the increased brightness are less problematic. However, the image sensor of the camera may also be adjusted to compensate for the additional brightness and/or the acquired images may be corrected for the additional brightness.

In some examples, the brightness of the light source may be increased at any time where the display becomes inactive or an error with the display is detected of the video laryngoscope is detected. For instance, if the video laryngoscope detects that the display screen and/or components (e.g., camera, image processing components, etc.) that allow the captured images to be displayed have malfunctioned, the brightness of the light source may be increased to allow for more effect direct laryngoscopy. When a battery level of the video laryngoscope drops below a threshold level, the display screen may be deactivated to conserve power and the brightness of the light source may be increased to facilitate direct laryngoscopy.

At operation 612, a recording of the training session is stored, and the recording may be automatically tagged as a training session. The recording includes the images from the camera acquired during the training session. The additional sensor data may also be stored as part of a recording of the training session. For example, the acquired images along with the sensor data may be stored together as a recording of the training session.

At operation 614, an indication to operate the video laryngoscope in a video laryngoscopy (VL) mode is received. The indication may be an indication to exit the DL training mode. The indication may be any or all input at the video laryngoscope (e.g., any touch at the integrated display, any press of a power button, and/or other input). Inclusivity of any user input with the video laryngoscope to exit or end the DL mode (e.g., to operate in VL mode) may reduce the likelihood of an operator being unintentionally stuck in DL mode. Alternatively, the indication may be a specific input at the video laryngoscope, such as a swipe at the integrated display screen (e.g., such as to peel back a digital mask or filter applied to the acquired images at operation 606).

At operation 616, the unobscured acquired images are displayed on the display of the video laryngoscope. Display of the unobscured acquired images may be in response to receiving the indication to operate the video laryngoscope in the VL mode. Sending of the acquired images to an external display and/or external device may be stopped or continued. The unmodified acquired images may be shown in place of the modified images and/or modification of the acquired images may be stopped in response to receiving the indication to operate the video laryngoscope in the VL mode. The recording of the training session may also continue, such that that recording of the training session includes acquired images during the VL mode as well. The brightness of the light source may also be reduced when returning to the VL mode from the DL training mode.

The techniques introduced above may be implemented for a variety of medical devices or devices where direct and indirect views are possible. A person of skill in the art will understand that the technology described in the context of a video laryngoscope for human patients could be adapted for use with other systems such as laryngoscopes for non-human patients or medical video imaging systems.

Those skilled in the art will recognize that the methods and systems of the present disclosure may be implemented in many manners and as such are not to be limited by the foregoing aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for operating a video laryngoscope in a direct laryngoscopy training mode, the method comprising:
acquiring images using a camera of the video laryngoscope;
receiving an indication to operate the video laryngoscope in a direct laryngoscopy mode;
in response to receiving the indication to operate the video laryngoscope in the direct laryngoscopy training mode, applying displaying obscured acquired images on an integrated display of the video laryngoscope;
receiving an indication to operate the video laryngoscope in a video laryngoscopy mode; and
in response to receiving the indication to operate the video laryngoscope in the video laryngoscopy mode, displaying, on the integrated display of the video laryngoscope, the acquired images.

2. The method of claim 1, wherein the obscured acquired images include the acquired images and one or more of the following modifications:
a blur;
a watermark; or
overlaid text.

3. The method of claim 1, further comprising:
in response to receiving the indication to operate the video laryngoscope in the video laryngoscopy mode, reducing a brightness of a light source of the video laryngoscope.

4. The method of claim 1, wherein the indication to operate the video laryngoscope in the video laryngoscopy mode is any input at the integrated display of the video laryngoscope.

5. The method of claim 1, further comprising:
recording the acquired images in a recording; and
labelling at least a portion of the recording as a training session.

6. The method of claim 1, further comprising:
displaying, at the integrated display of the video laryngoscope, a torque indicator associated with an amount of torque detected by the video laryngoscope.

7. A video laryngoscope training mode system, comprising:
an external display; and
a video laryngoscope comprising:
an integrated display;
a camera that acquires images while the video laryngoscope is powered on;
a processor that operates to:
receive an indication to operate the video laryngoscope in a direct laryngoscopy training mode;
in response to receiving the indication to operate the video laryngoscope in the direct laryngoscopy training mode, display obscured acquired images on an integrated display of the video laryngoscope; and
transmit the acquired images to the external display for unobscured display of the acquired images, concurrently with displaying the filtered images at the integrated display.

8. The system of claim 7, the video laryngoscope further comprising a light source with an adjustable brightness.

9. The system of claim 8, wherein the processor further operates to increase the brightness of the light source in response to receiving the indication to operate the video laryngoscope in the direct laryngoscopy training mode.

10. The system of claim 7, wherein the video laryngoscope further comprises a torque sensor; and the processor further operates to:
acquire torque data from the torque sensor; and
transmit the torque data to the external display for display.

11. The system of claim 7, wherein the acquired images are stored in memory of the video laryngoscope, and wherein at least a portion of the acquired images are labeled a training session.

12. A video laryngoscope, comprising:
a power switch that powers the video laryngoscope on and off in response to user input;
a handle portion;
a display screen coupled to the handle portion;
a blade portion, coupled to the handle portion, configured to be inserted into a mouth of a patient;
a camera, positioned at a distal end of the blade portion, that acquires images while the video laryngoscope is powered on;
a light source positioned at the distal end of the blade portion;
a processor that operates to:
activate a direct laryngoscopy training mode;
receive acquired images from the camera;
in response to activating the direct laryngoscopy training mode:
display obscured acquired images on the display screen; and
increase a brightness or intensity of the light source.

13. The video laryngoscope of claim 12, wherein the processor operates to:
transmit the acquired images to an external display for unobscured display of the acquired images.

14. The video laryngoscope of claim 12, wherein activating the direct laryngoscopy training mode is based on receiving a long press of the power switch.

15. The video laryngoscope of claim 12, wherein while in the direct laryngoscopy training mode, the processor operates to display, on the display screen, unobscured device information.
